(19) United States
(12) Patent Application Publication
(10) Pub. No.: US 2018/0214559 A1
(43) Pub. Date: Aug. 2, 2018

(54) NOVEL DRUG DELIVERY CONJUGATED MOIETY FOR ORAL ADMINISTRATION OF DRUG UNSUITABLE FOR ORAL ADMINISTRATION AND PREPARATION METHOD THEREOF

(71) Applicant: ST PHARM CO., LTD., Siheung-si (KR)

(72) Inventors: Kyungjin KIM, Siheung-si (KR); Jisuk YUN, Siheung-si (KR); Hyukjun JUNG, Siheung-si (KR); Myungyun LEE, Siheung-si (KR); Hwajung NAM, Siheung-si (KR); Ok-cheol JEON, Seoul (KR)

(21) Appl. No.: 15/746,133

(22) PCT Filed: Jul. 20, 2015
(86) PCT No.: PCT/KR2015/007516

§ 371 Date: Jan. 19, 2018

(51) Int. Cl. A61K 47/54; A61K 9/00; A61K 38/28; A61K 31/727

(52) U.S. Cl. CPC A61K 47/554; A61K 31/727; A61K 38/28; A61K 9/0053

(57) ABSTRACT

The present invention provides a novel drug delivery conjugated moiety for oral administration of a drug that is not suitable for oral administration or a pharmaceutically acceptable salt thereof. When the drug delivery conjugated moiety of the present invention or a pharmaceutically acceptable salt thereof is combined with a drug, which is not suitable for oral administration, and is administered orally, it exhibits an excellent absorption rate without decreasing the biological activities of the drug. Moreover, the drug delivery conjugated moiety of the present invention or a pharmaceutically acceptable salt thereof can be easily prepared in a few steps, which is very advantageous in terms of mass production.

### TECHNICAL FIELD

The present invention relates to a novel drug delivery conjugated moiety for oral administration of drugs that are not suitable for oral administration or a pharmaceutically acceptable salt thereof, a preparation method thereof, a pharmaceutical composition comprising the same, and a treatment method using the same.

### BACKGROUND ART

Oral administration is the most ideal route among various routes for administering drugs to animals including human beings; however, most drugs, when administered orally, exhibit a very limited absorption rate due to various physical, chemical, and biological barriers, and thus efficient delivery of drugs is difficult to achieve. Drugs that are not suitable for oral administration may include biologically active peptides such as insulin, calcitonin, growth hormones, and glucagon-like peptide-1; mucopolysaccharides and polysaccharides including heparin and heparinoid; antibiotics; and other organic materials.

For example, heparin is a polysaccharide composed of sulfated D-glucosamine and L-iduronic acid residues and has many physiological roles such as anticoagulant activity, inhibition of smooth muscle cell proliferation, etc. In particular, heparin is a useful anticoagulant agent that interacts strongly with antithrombin III to prevent the formation of fibrin clots. Due to these properties, heparin has been widely used for the treatment of deep vein thrombosis (DVT) and pulmonary embolism (PE). Despite these physiological usefulness of heparin, heparin is not absorbed efficiently from the gastrointestinal tract, nasal or buccal mucosal layers, and the like due to its large molecular weight and strong negative charge. Therefore, the only routes of administration used clinically are intravenous and subcutaneous injections.

Moreover, insulin is produced by Langerhans beta cells of the pancreas and released from the pancreas, when the blood glucose level is high, to regulate the glucose level. Due to these properties, insulin has been widely used as an antidiabetic agent. However, due to its large molecular weight, insulin is not suitable for oral administration.

Therefore, there is a need to develop a novel drug delivery conjugated moiety which, when administered orally with a drug that is not suitable for oral administration, exhibits an excellent absorption rate without decreasing the biological activities of the drug and can be easily prepared, and thus the present inventors have made the present invention after a long study.

### DISCLOSURE

### Technical Problem

An object of the present invention is to provide a novel drug delivery conjugated moiety or a pharmaceutically acceptable salt thereof which, when administered orally with a drug that is not suitable for oral administration, exhibits an excellent absorption rate without decreasing the biological activities of the drug and can be easily prepared, and a preparation method thereof.

Moreover, another object of the present invention is to provide a pharmaceutical composition comprising a novel drug delivery conjugated moiety or a pharmaceutically acceptable salt thereof and a treatment method using the same.

### Technical Solution

In order to accomplish the objects of the present invention, the present invention provides a novel drug delivery conjugated moiety or a pharmaceutically acceptable salt thereof, a preparation method thereof, a pharmaceutical composition comprising the same, and a treatment method using the same, which will be described in detail below.

Drug Delivery Conjugated Moiety or Pharmaceutically Acceptable Salt Thereof

The present invention provides a novel drug delivery conjugated moiety represented by the following Formula I or a pharmaceutically acceptable salt thereof:

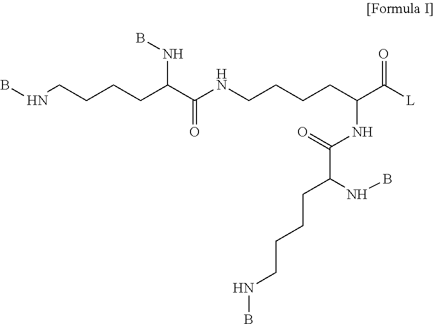

wherein B is a bile acid residue and L is a linker.

When the drug delivery conjugated moiety of the present invention or a pharmaceutically acceptable salt thereof is combined with a drug, which is not suitable for oral administration, and is administered orally, it exhibits an excellent absorption rate without decreasing the biological activities of the drug. Therefore, the drug delivery conjugated moiety of the present invention or a pharmaceutically acceptable salt thereof can be very effectively used as a drug delivery conjugated moiety for oral administration of a drug that is not suitable for oral administration due to its low absorption rate.

Moreover, the drug delivery conjugated moiety of the present invention or a pharmaceutically acceptable salt thereof can be easily prepared in a few steps, which is very advantageous in terms of mass production and very useful in industrial applications.

In the present invention, the drug that is not suitable for oral administration may be a polypeptide or polysaccharide biologically active agent.

According to an embodiment of the present invention, the drug may be a polysaccharide and may preferably have a molecular weight of more than 1000 Da. Examples of the polysaccharide include heparin, heparin sodium, sulfonated polysaccharide, cellulose, hydroxymethyl cellulose, and hydroxypropyl cellulose. Preferably, heparin having anticoagulant activity may be selected. Heparin is an acidic mucopolysaccharide composed of repeating units of D-glucosamine and L-iduronic acid and may be selected from the group consisting of high molecular weight heparins (HMWH), low molecular weight heparins (LMWH), heparin fragments, recombinant heparins, heparin analogues, heparin sulfates, and sulfonated polysaccharides having heparin activities, and the most preferred is low molecular weight heparins (LMWH).

Meanwhile, the polysaccharide including heparin may have a carbonyl group at its end site. The polysaccharide having a carbonyl group at its end site can be connected to a linker (L) having an amine group by reductive amination reaction.

Specifically, a hydroxyl group located at the end site of cellulose can be oxidized to ketone, which can be then connected to the amine group of the linker. Moreover, an aldehyde group located at the end site of heparin can be connected to the amine group of the linker (L).

According to another embodiment of the present invention, the drug may be a polypeptide. The polypeptide is a polymer composed of more than 10 amino acid residues linked by peptide bonds and may preferably have a molecular weight of more than 1000 Da in the present invention. Examples of the polypeptide may include insulin, insulinotropic peptide or calcitonin. Moreover, the insulinotropic peptide may be selected from the group consisting of GLP-1, Exendin-3, Exendin-4, and agonists, derivatives, and fragments thereof. Meanwhile, the polypeptide may comprise a cysteine residue at the N-terminus or C-terminus. The cysteine residue of the polypeptide may have a naturally-occurring form or a modified form (e.g., substitution or addition). A thiol group of the cysteine residue located at the N-terminus or C-terminus can be connected to a linker (L) having a maleimide group, an iodoacetamide group, or a disulfide group.

Moreover, in the present invention, the bile acid residue (B) is coupled to ASBT (Apical Sodium dependent Bile acid Transporter) in the enterocyte membrane and is responsible for vesicular transport, and four bile acid residues are incorporated in the drug delivery conjugated moiety, which significantly increases the absorption rate of the drug. For example, the bile acid may be selected from the group consisting of cholic acid, deoxycholic acid, chenodeoxycholic acid, lithocholic acid, ursocholic acid, ursodeoxycholic acid, isoursodeoxycholic acid, lagodeoxycholic acid, glycocholic acid, taurocholic acid, glycodeoxycholic acid, glycochenodeoxycholic acid, dehydrocholic acid, hyocholic acid, and hyodeoxycholic acid residues. The bile acid may preferably be deoxycholic acid.

Moreover, in a complex of the present invention, four bile acid residues may be identical to or different from each other. However, more preferred are those identical to each other for manufacturing convenience.

Furthermore, the drug delivery conjugated moiety of the present invention or a pharmaceutically acceptable salt thereof comprises a linker (L) that couples the drug delivery conjugated moiety with the drug. In the present invention, the linker has a functional group that can be coupled to the drug, preferably a functional group that can be coupled to an end site of the drug. In the present invention, the functional group of the linker may vary depending on the type of the functional group located at the end site of the drug. For example, to form a thioester bond by reaction with the thiol group of the cysteine residue of the polypeptide, the functional group of the linker may be selected from the group consisting of maleimide, iodoacetamide or disulfide group, but not limited thereto. Moreover, to form a bond by reductive amination of a carbonyl group such as an aldehyde group or ketone group of the polysaccharide, the functional group of the linker may be an amine group, but not limited thereto. Examples of the linker include an alkyl chain, polyethyleneglycol (PEG), pentaethylenehexamine, 1,5-diamino-2-methylpentane, and ethylenediamine (EDA) residue.

As used herein, the term “pharmaceutically acceptable salt” refers to a salt that is conventionally used in the pharmaceutical industry, and examples of the pharmaceutically acceptable salt include salts of inorganic ions such as sodium, potassium, calcium, magnesium, lithium, copper, manganese, zinc, iron, etc.; salts of inorganic acids such as hydrochloric acid, phosphoric acid, sulfuric acid, etc.; salts of organic acids such as ascorbic acid, citric acid, tartaric acid, lactic acid, maleic acid, malonic acid, fumaric acid, glycolic acid, succinic acid, propionic acid, acetic acid, orotic acid, acetylsalicylic acid, etc.; and salts of amino acids such as lysine, arginine, guanidine, etc. Moreover, examples of the pharmaceutically acceptable salt include salts of organic ions such as tetramethylammonium, tetraethylammonium, tetrapropylammonium, tetrabutylammonium, benzyltrimethylammonium, benzethonium, etc. which can be used in pharmaceutical reaction, purification, and isolation processes. However, the types of salts as referred to herein are not limited to the listed salts.

Preparation Method of Drug Delivery Conjugated Moiety or Pharmaceutically Acceptable Salt Thereof

Moreover, the present invention provides a preparation method of the above-mentioned novel drug delivery conjugated moiety or a pharmaceutically acceptable salt thereof.

Specifically, the preparation method of the drug delivery conjugated moiety of the present invention or a pharmaceutically acceptable salt thereof may comprise the steps of: (S1) preparing a compound of the following Formula 1 and a compound of the following Formula 2 from lysine; (S2) preparing a compound of the following Formula 3 by reaction of the compound of Formula 1 with the compound of Formula 2; (S3) preparing a compound of the following Formula 4 by deprotection of amine protecting groups of Formula 3; (S4) preparing a compound of the following Formula 5 by reaction of the compound of Formula 4 with bile acids; and (S5) preparing a compound of the following Formula I by connecting a linker to the compound of Formula 5:

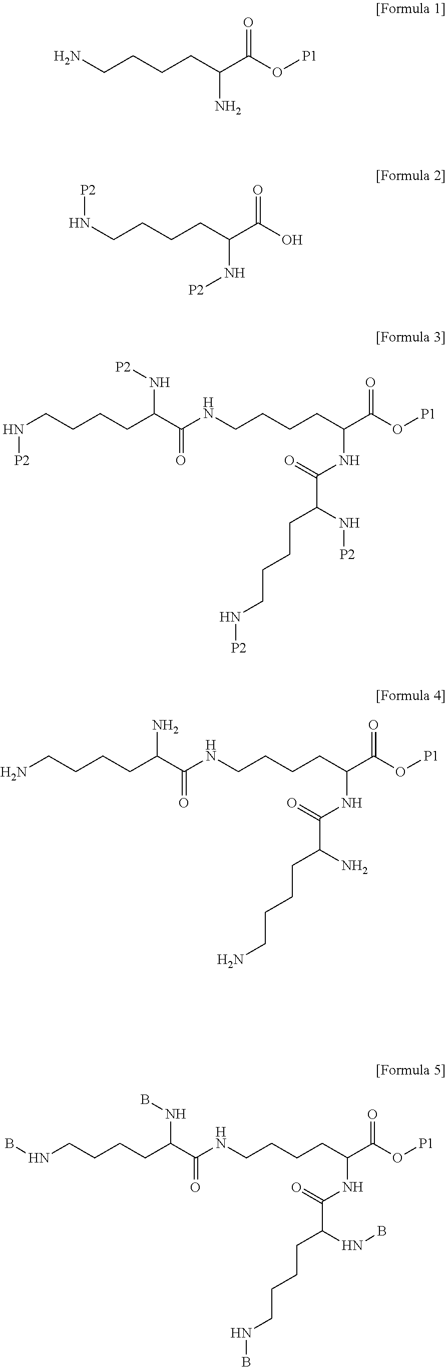

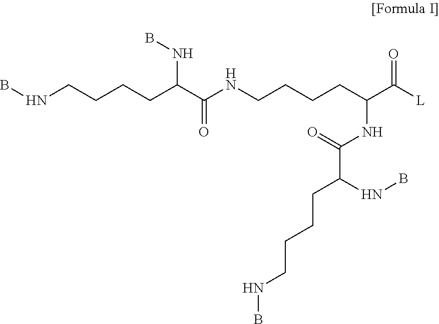

wherein B and L are the same as mentioned above; P1 is a carboxyl protecting group; and P2 is an amine protecting group.

The starting material in the step (S1) is lysine, and more preferred is L-lysine. Lysine is an amino acid having two amine groups and is very advantageous because it facilitates the preparation of the drug delivery conjugated moiety of the present invention.

The compound of Formula 1, a product in the step (S1), has a structure in which one carboxyl group of the lysine is protected. The P1 may be any one of carboxylic acid protecting groups. The P1 may preferably be C₁-C₆ alkyl or benzyl, more preferably methyl, but not limited thereto. The protection of carboxylic acid groups can be performed under protection reaction conditions conventionally used in the art.

Moreover, the compound of Formula 2, another product in the step (S1), has a structure in which both amine groups of the lysine are protected. The P2 may be any one of amine protecting groups. The P2 may preferably be Boc, Cbz, Moz or Fmoc, more preferably Boc, but not limited thereto. The protection of amine groups can be performed under protection reaction conditions conventionally used in the art.

In the step (S2), a lysine trimer of Formula 3 is prepared by coupling the amine groups of the compound of Formula 1 to the carboxyl group of the compound of Formula 2. The compound of Formula 2 may preferably be used in an amount of 2.0 to 3.0 equivalents relative to the compound of Formula 1. The reaction in the step (S2) can be performed under peptide coupling reaction conditions conventionally used in the art, but not limited thereto.

In the step (S3), the compound of Formula 4 is prepared by deprotection of the amine protecting groups of Formula 3. This step can be performed under conditions for deprotection of amine protecting groups conventionally used in the art.

In the step (S4), the compound of Formula 5 is prepared by reaction of the compound of Formula 4 with bile acids. Specifically, the amine groups of the compound of Formula 4 and the carboxyl group of the bile acid are condensed. This step can be performed under peptide coupling conditions conventionally used in the art. The bile acid used in the step (S4) is the same as described above.

In the step (S5), the drug delivery conjugated moiety of Formula I is prepared by connecting a linker to the compound of Formula 5. This step may vary depending on the type of the linker to be connected. For example, an amine functional group may be introduced by reaction of the compound of Formula 5 with ethylenediamine (EDA). In this case, the step (S5) can be performed under conditions for amidation of ester conventionally used in the art.

As such, the drug delivery conjugated moiety of the present invention or a pharmaceutically acceptable salt thereof can be prepared in a few steps by the preparation method of the present invention. Therefore, the preparation method of the present invention is very advantageous in terms of mass production, and the drug delivery conjugated moiety of the present invention prepared using the same is very useful in industrial applications.

Pharmaceutical Composition Comprising the Drug Delivery Conjugated Moiety of the Present Invention or Pharmaceutically Acceptable Salt Thereof, Use Thereof, and Treatment or Prevention Method Using the Same

The present invention provides a pharmaceutical composition comprising: the drug delivery conjugated moiety of the present invention or a pharmaceutically acceptable salt thereof; and a polypeptide or polysaccharide biologically active agent.

In the pharmaceutical composition of the present invention, its medical use may vary depending on the type of the biologically active agent. That is, the pharmaceutical composition of the present invention can be used in connection with the known medical use of the biologically active agent. For example, when the biologically active agent is insulin, the composition of the present invention can be used for the prevention or treatment of diabetes. Moreover, when the biologically active agent is heparin, the composition of the present invention can be used as an anticoagulant agent, can be used for the prevention or treatment of cancer or inflammatory disease, and can preferably be used as an anticoagulant agent.

In the composition of the present invention, the linker (L) of the drug delivery conjugated moiety or a pharmaceutically acceptable salt thereof may be connected to the end site of the polypeptide or polysaccharide biologically active agent to form a complex, which can be administered orally.

Moreover, the pharmaceutical composition of the present invention may further comprise a solubilizer. The solubilizer comprises both hydrophilic and hydrophobic molecules, thereby preventing self-aggregation of the complex of the present invention. Specifically, a hydrophilic part of the solubilizer interacts with the biologically active agent such as heparin, while a hydrophobic part interacts with a bile acid moiety to reduce the surface tension. Through this action of the solubilizer, the complex of the present invention interacts with a bile acid transporter, which leads to a higher absorption rate of the complex. The type of the solubilizer is not limited as long as it can facilitate the effective intestinal absorption of the complex of the present invention. Examples of the solubilizer include polyethylene oxide, hydroxyalkyl cellulose, hydroxypropylalkyl cellulose, polyvinyl alcohol, polyvinylpyrrolidone, copovidone, sodium carboxymethyl cellulose, carbopol, sodium alginate, xanthan gum, locust bean gum, glycofurol, poloxamer, cyclodextrin or surfactant, but not limited thereto.

Examples of the surfactant include anionic surfactants, non-ionic surfactants, zwitterionic surfactants or mixtures thereof.

The pharmaceutical composition of the present invention may further comprise pharmaceutically acceptable additives such as a diluent, a binder, a disintegrant, and a lubricant, as long as the effects of the present invention are not impaired.

Examples of the diluent include sugar, starch, microcrystalline cellulose, lactose (lactose hydrate), glucose, D-mannitol, alginate, alkaline earth metal salt, clay, polyethylene glycol, anhydrous dibasic calcium phosphate or mixtures thereof.

Examples of the binder include starch, microcrystalline cellulose, highly dispersive silica, mannitol, D-mannitol, sucrose, lactose hydrate, polyethylene glycol, polyvinylpyrrolidone (povidone), polyvinylpyrrolidone copolymer (copovidone), hypromellose, hydroxypropylcellulose, natural gum, synthetic gum, gelatin or mixtures thereof.

Examples of the disintegrant include starches or modified starches such as sodium starch glycolate, corn starch, potato starch or pregelatinized starch, etc.; clays such as bentonite, montmorillonite or veegum, etc.; celluloses such as microcrystalline cellulose, hydroxypropylcellulose or carboxymethylcellulose, etc.; algins such as sodium alginate or alginic acid, etc.; crosslinked celluloses such as croscarmellose sodium, etc.; gums such as guar gum, xanthan gum, etc.; crosslinked polymers such as crosslinked polyvinylpyrrolidone (crospovidone), etc.; effervescent agents such as sodium bicarbonate, citric acid, etc.; or mixtures thereof.

Examples of the lubricant include talc, stearic acid, magnesium stearate, calcium stearate, sodium lauryl sulfate, hydrogenated vegetable oil, sodium benzoate, sodium stearyl fumarate, glyceryl behenate, glyceryl monolaurate, glyceryl monostearate, glyceryl palmitostearate, colloidal silicon dioxide or mixtures thereof.

For oral administration, the pharmaceutical composition of the present invention may be formulated into solid dosage forms such as tablets, pills, powders, granules or capsules, etc., and these solid dosage forms may be prepared by mixing the complex with one or more excipients such as starch, calcium carbonate, sucrose or lactose, gelatin, etc. Moreover, lubricants such as magnesium stearate, talc, etc. can be used in addition to simple excipients. Furthermore, the pharmaceutical composition may be formulated into liquid dosage forms such as suspensions, liquid for internal use, emulsions, syrups, etc., and various excipients such as humectants, sweeteners, aromatics, preservatives, etc. in addition to water and liquid paraffin can be used for the formulation of liquid dosage forms.

The pharmaceutical composition of the present invention may be administered once or several times a day at regular intervals considering pharmacologically effective dose of the biologically active agent. However, the dose of the complex can be appropriately adjusted depending on the condition of a patient, such as the patient's severity, age, sex, weight, etc., and the drug's dosage form, administration route, and administration period of the drug.

Moreover, the pharmaceutical composition according to the present invention can be used in combination with other active ingredients having the same effect as the selected biologically active agent.

Furthermore, the pharmaceutical composition according to the present invention can be used alone or in combination with various methods such as hormone therapy, drug therapy, etc.

In addition, the present invention provides a use of a composition comprising a novel drug delivery conjugated moiety of the present invention or a pharmaceutically acceptable salt thereof, for preparation of an antidiabetic agent or an anticoagulant agent.

Moreover, the present invention provides a treatment or prevention method comprising administrating a composition comprising a drug delivery conjugated moiety of the present invention or a pharmaceutically acceptable salt thereof to a subject in need thereof.

The composition used in the above method comprises the pharmaceutical composition described herein.

Moreover, in the treatment or prevention method of the present invention, the subject that needs the composition of the present invention comprises a mammal, preferably a human being.

A disease to which the treatment or prevention method of the present invention is applied may vary depending on the type of the biologically active agent. That is, the pharmaceutical composition of the present invention can be used in connection with the known medical use of the biologically active agent. For example, when the biologically active agent is insulin, the composition of the present invention can be used for the prevention or treatment of diabetes. Moreover, when the biologically active agent is heparin, the composition of the present invention can be used for the prevention or treatment of anticoagulant activity-associated disease, cancer or inflammatory disease.

### Advantageous Effects

When the drug delivery conjugated moiety of the present invention or a pharmaceutically acceptable salt thereof is combined with a drug, which is not suitable for oral administration, and is administered orally, it exhibits an excellent absorption rate while maintaining the biological activities of the drug. Moreover, the drug delivery conjugated moiety of the present invention or a pharmaceutically acceptable salt thereof can be easily prepared in a few steps, which facilitates mass production.

### MODE FOR INVENTION

Hereinafter, preferred examples will be provided for better understanding of the present invention. However, the following examples are provided only for illustrating the present invention, and the scope of the present invention is not limited thereto.

### Example 1: Preparation of Drug Delivery Conjugated Moiety of the Present Invention

Step 1: Protection of Lysine

Preparation of Compound of Formula 1

To a solution of 2,2-dimethoxypropane (70 mL) and conc. hydrochloric acid (18.0 mL) in methanol (110 mL) was added L-lysine (10 g, 54.75 mmol). The reaction mixture was heated to reflux for 3 hours, cooled down to room temperature and stirred overnight. The mixture was concentrated under reduced pressure to afford L-lysine methyl ester dihydrochloride as a white solid (9.88 g, 42.38 mmol).

Preparation of Compound of Formula 2

To a solution of L-lysine (20 g, 109.5 mmol) in 160 mL of water and 160 mL of tetrahydrofuran was added sodium carbonate (24 g, 226.4 mmol). After stirring for 15 minute, the reaction mixture was cooled down to 0° C. and di-tert-butyl-dicarbonate (48.93 g, 224.4 mmol) was slowly added. After stirring overnight at room temperature, the reaction mixture was diluted with ethyl acetate (66.7 mL) and 6N hydrochloric acid solution (56.7 mL) was added to adjust pH to 3 or lower. The aqueous layer was extracted with ethyl acetate. The combined organic layer was dried over sodium sulfate and concentrated under reduced pressure to provide the desired product (34.2 g, 98.8 mmol).

Step 2: Preparation of Lysine Trimer (Formula 3)

To a solution of L-lysine methyl ester dihydrochloride (1 g, 4.3 mmol) prepared in step 1 in ethyl acetate (20 mL) was slowly added triethylamine (0.9 g, 8.9 mmol). After stirring for 10 minutes at room temperature, di-boc lysine (2.6 g, 7.4 mmol) prepared in step 1 and N-hydroxysuccinimide (0.9 g, 7.4 mmol) were added. To the reaction mixture dicyclohexylcarbodimide (1.5 g, 7.4 mmol) was added at 0° C.

After stirring overnight, the reaction mixture was cooled down to 0° C., filtered to remove precipitate. The organic layer was washed with saturated sodium bicarbonate solution, 12% sodium bisulfate solution, saturated sodium bicarbonate solution and brine. The organic layer was dried over sodium sulfate and concentrated under reduced pressure. The residue was purified by silica gel column chromatography (dichloromethane/methanol/triethylamine=95/5/0.1) to provide the desired product (2.3 g, 2.8 mmol).

Step 3: Deprotection of Amine Groups (Preparation of Compound of Formula 4)

A mixture of methanol (198 mL) and acetyl chloride (15.6 g, 0.2 mol) was stirred for 1 hour at 0° C. To the reaction mixture was slowly added lysine trimer (7.8 g, 9.55 mmol) prepared in step 2. The reaction mixture was allowed to warm up to room temperature slowly and then stirred overnight. The reaction mixture was concentrated under reduced pressure to afford the desired product Formula 4 (5.04 g, 8.97 mmol).

Step 4: Binding of Bile Acids (Preparation of Compound of Formula 5)

To a solution of amine-deprotected compound (5.04 g, 8.97 mmol) obtained in step 3 in methanol (25 mL) and dimethylformamide (151 mL) was added slowly N-methylmorpholine (10.87 g, 107 mmol) over 0.5 hours. After stirring for 1 hour, the reaction mixture was cooled down to 5° C. To the reaction mixture was slowly added N-succinimidyl deoxycholic ester (19.75 g, 40.3 mmol) dissolved in 92 mL of dimethylformamide. The reaction mixture was allowed to warm up to room temperature and stirred overnight. The reaction solution was concentrated under reduced pressure and the residue was purified by silica gel column chromatography (dichloromethane/methanol=9/1) to provide the desired product (10.08 g, 5.26 mmol).

Step 5: Connection of Linker (Preparation of Drug Delivery Conjugated Moiety of Formula I)

To a solution of Formula 5 (5 g, 2.61 mmol) obtained in step 4 in ethanol (40 mL) was slowly added ethylenediamine (21.3 g, 0.35 mol) at 5° C. or lower. The reaction mixture was allowed to warm up to room temperature and stirred for 3 days. The reaction mixture was concentrated under reduced pressure and the residue was purified by silica gel column chromatography (chloroform/methanol/ammonia solution=8/2/0.25) to afford the desired product Formula I (2.54 g, 1.31 mmol).

### Example 2: Preparation of Drug Delivery Conjugated Moiety-Heparin Complex of the Present Invention

To a solution of enoxaparin (50 mg, 11 μM) in a mixture of H₂O/DMF (1/7 mL) was added drug delivery conjugated moiety prepared in Example 1 (130 mg, 0.067 mmol). After stirring at 60° C. for 24 hours, sodium cyanoborohydride (7.0 mg, 0.11 mmol) was added. After stirring for 4 hours, the reaction mixture was diluted with ethanol and the precipitate was filtered to provide the desired product as an off-white powder.

### Experimental Example 1: Measurement of PK Parameters

The following experiment was performed to determine the pharmacokinetic (PK) behavior of heparin administered orally using the drug delivery conjugated moiety of the present invention.

Prior to drug administration, experimental animals (rats) were fasted for more than 4 hours to empty the stomach. Then, after the dorsal skin was fixed, forced oral administration was performed using a sonde and a syringe for oral administration, and the animals were fed again about 4 hours after the administration. The drug delivery conjugated moiety-heparin complex prepared in Example 2 was administered orally, and blood samples were collected over 0.25, 0.5, 1, 2, 3, 4, 6, 8 hours. Blood samples (450 μL) were collected in a tube containing sodium citrate (50 μL) and centrifuged at 4500×g for 20 minutes to isolate plasma, which was then stored in a deep freezer at −70° C. The concentration of the drug delivery conjugated moiety-heparin complex in the plasma was analyzed using the COATEST HEPARIN FXa assay (Chromogenix). The results are shown in the following table 1:

TABLE 1

| | | | | | | | |
|---|---|---|---|---|---|---|---|
| | Dose | Eₘₐₓ^{a)} | Tₘₐₓ^{b)} | AUC^{c)} | t_{1/2}^{d)} | CL^{e)} | F^{f)} |
| Substances | (mg/kg) | (IU/ml) | (h) | (IU · h/ml) | (h) | (ml/h/kg) | (%) |

Intravenous administration

| | | | | | | | |
|---|---|---|---|---|---|---|---|
| LMWH | 2 | 1.61 ± 0.02 | 0.2 ± 0.0 | 1.8 ± 0.1 | 1.2 ± 0.1 | 0.7 ± 0.0 | — |

Oral administration

| | | | | | | | |
|---|---|---|---|---|---|---|---|
| Example 2 | 5 | 0.46 ± 0.04 | 1.0 ± 0.0 | 1.99 ± 0.4 | 2.3 ± 0.6 | 2.4 ± 0.5 | 44.1 ± 9.0 |

^{a)}Maximum effective concentration;
^{b)}Time to reach maximum effective concentration;
^{c)}Area under the concentration-time curve from 0 to 8 h;
^{d)}Half-life of drug;
^{e)}Clearance; and
^{f)}Absolute bioavailability.

The oral administration of the complex of Example 2 to rats showed an excellent bioavailability of about 40% or higher, compared to the intravenous administration of low molecular weight heparin (LMWH).

### Experimental Example 2: Measurement of FXa

The term “heparin activity” refers to the anticoagulant ability of heparin. The COATEST HEPARIN FXa assay kit from Chromogenix was used to determine the anticoagulant activity of heparin that was administered orally using the drug delivery conjugated moiety of the present invention.

Stock solutions were prepared from 10 IU/ml of standard solution by accurately preparing test substances and then diluted to 0.1 IU/ml with buffer working solution (Tris 0.5 mol/L, pH=8.4, 10 ml, Chromogenix) to prepare standard solutions for each dose as shown in the following table 2 and used for the analysis.

TABLE 2

| | Heparin | Heparin | Buffer | | |
|---|---|---|---|---|---|
| Standard | IU/ml | dilution | working | Normal | |
| solution | Plasma | 0.1 IU/ml | solution | Plasma | Antithrombin |
| Standard | 0.1 | 100 μl | 700 μl | 100 μl | 100 μl |
| solution A | | | | | |
| Standard | 0.3 | 300 μl | 500 μl | 100 μl | 100 μl |
| solution B | | | | | |
| Standard | 0.5 | 500 μl | 300 μl | 100 μl | 100 μl |
| solution C | | | | | |
| Standard | 0.7 | 700 μl | 100 μl | 100 μl | 100 μl |
| solution D | | | | | |

Each 200 μL of pretreated samples was placed in a cuvette and preheated at 37° C. for 3 to 4 minutes. Each 100 μl of FXa (Bovine Factor Xa 71 nkat., Chromogenix) was added thereto and left at 37° C. for about 30 seconds. Then, each 200 μl of S-2222 (Chromogenic substrate (Bz-Ile-Glu-(g-OR)-Gly-Arg-pNA.HCl), Chromogenix) was added thereto and left at 37° C. for 3 minutes. Subsequently, each 300 μl of 20% acetic acid was added thereto, and then the absorbance was measured at 405 nm. At this time, the measurement should be performed within 4 hours.

A linear regression equation was obtained using calibration curve of the standard solutions and converted to obtain the titers of the samples. The results of the heparin activities (FXa) are shown in the following table 3. The complex of Example 2 administered orally showed almost the same activities as the low molecular weight heparin (LMWH) administered intravenously.

TABLE 3

| | Anti-factor Xa activity | |
|---|---|---|
| LMWH | 101 | IU/mg |
| Example 2 | 101.1 | IU/mg |

### Experimental Example 3: Measurement of DVT Activities

The following experiment was performed to determine the effects of heparin administered orally using the drug delivery conjugated moiety of the present invention on deep vein thrombosis (DVT).

Low molecular weight heparin (enoxaparin) was administered to one group by subcutaneous injection, and the formulated complex of Example 2 was administered orally to other groups. The animals were anesthetized by abdominal injection of ketamine (45 mg/kg) and xylazine (5 mg/kg) and subjected to surgery to open the abdominal cavities of the rats, and the superior vena cava and inferior vena cava were isolated. The distal end of the vein along about 3 cm was weakly tied off, and the remaining veins were strongly tied off. 60 minutes after the drug treatment, 1 mL/kg of human pooled plasma was administered intravenously to the ends of the tails at 37° C., and the veins were tied off after 15 seconds to prevent blood flow. The veins were isolated 120 minutes after the drug treatment and stored in a Petri dish with 3.8% sodium citrate. Then, thrombus was isolated, and the amount of plasma generated was measured.

The comparison of the effects of the bile acid tetramer-biologically active agent complex on DVT disease models showed that when the complex of Example 2 was administered at a dose of 5 mg/kg, about 25% thrombus was generated, compared to the group to which the drug was not treated, which was almost the same results as the group to which LMWH was injected subcutaneously.

### INDUSTRIAL APPLICABILITY

When the drug delivery conjugated moiety of the present invention is combined with a drug, which is not suitable for oral administration, and is administered orally, it exhibits an excellent absorption rate while maintaining the biological activities of the drug. Moreover, the drug delivery conjugated moiety of the present invention can be easily prepared in a few steps, which facilitates mass production. Therefore, the drug delivery conjugated moiety of the present invention can be very effectively used for the oral administration of a drug that is not suitable for oral administration.

1. A drug delivery conjugated moiety represented by the following Formula I or a pharmaceutically acceptable salt thereof: 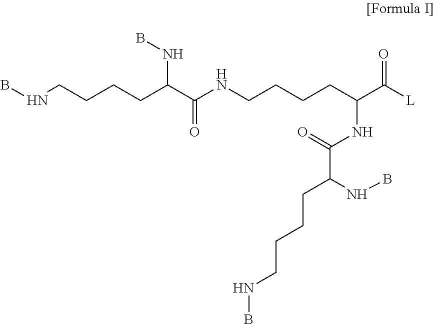
wherein B is a bile acid residue and L is a linker.

2. The drug delivery conjugated moiety or a pharmaceutically acceptable salt thereof of claim 1, wherein the drug delivery conjugated moiety is used for oral administration of a drug that is not suitable for oral administration.

3. The drug delivery conjugated moiety or a pharmaceutically acceptable salt thereof of claim 2, wherein the drug is a polypeptide or polysaccharide biologically active agent.

4. The drug delivery conjugated moiety or a pharmaceutically acceptable salt thereof of claim 3, wherein the drug is a polypeptide.

5. The drug delivery conjugated moiety or a pharmaceutically acceptable salt thereof of claim 4, wherein the polypeptide is an insulin or insulinotropic peptide.

6. The drug delivery conjugated moiety or a pharmaceutically acceptable salt thereof of claim 5, wherein the insulinotropic peptide is selected from the group consisting of GLP-1, Exendin-3, Exendin-4, and derivatives thereof.

7. The drug delivery conjugated moiety or a pharmaceutically acceptable salt thereof of claim 3, wherein the drug is a polysaccharide.

8. The drug delivery conjugated moiety or a pharmaceutically acceptable salt thereof of claim 7, wherein the polysaccharide is heparin.

9. The drug delivery conjugated moiety or a pharmaceutically acceptable salt thereof of claim 1, wherein the L comprises a functional group selected from the group consisting of a maleimide group, an iodoacetamide group, a disulfide group, and an amine group.

10. The drug delivery conjugated moiety or a pharmaceutically acceptable salt thereof of claim 1, wherein the L is coupled to an end site of a drug.

11. The drug delivery conjugated moiety or a pharmaceutically acceptable salt thereof of claim 1, wherein the B is a bile acid residue selected from the group consisting of cholic acid, deoxycholic acid, chenodeoxycholic acid, lithocholic acid, ursocholic acid, ursodeoxycholic acid, isoursodeoxycholic acid, lagodeoxycholic acid, glycocholic acid, taurocholic acid, glycodeoxycholic acid, glycochenodeoxycholic acid, dehydrocholic acid, hyocholic acid, and hyodeoxycholic acid residues.

12. A preparation method of a drug delivery conjugated moiety represented by the following Formula I or a pharmaceutically acceptable salt thereof, the method comprising the steps of:
(S1) preparing a compound of the following Formula 1 and a compound of the following Formula 2 from lysine;
(S2) preparing a compound of the following Formula 3 by reaction of the compound of Formula 1 with the compound of Formula 2;
(S3) preparing a compound of the following Formula 4 by deprotection of amine protecting groups of Formula 3;
(S4) preparing a compound of the following Formula 5 by reaction of the compound of Formula 4 with bile acids; and
(S5) preparing a compound of Formula I by connecting a linker to the compound of Formula 5: 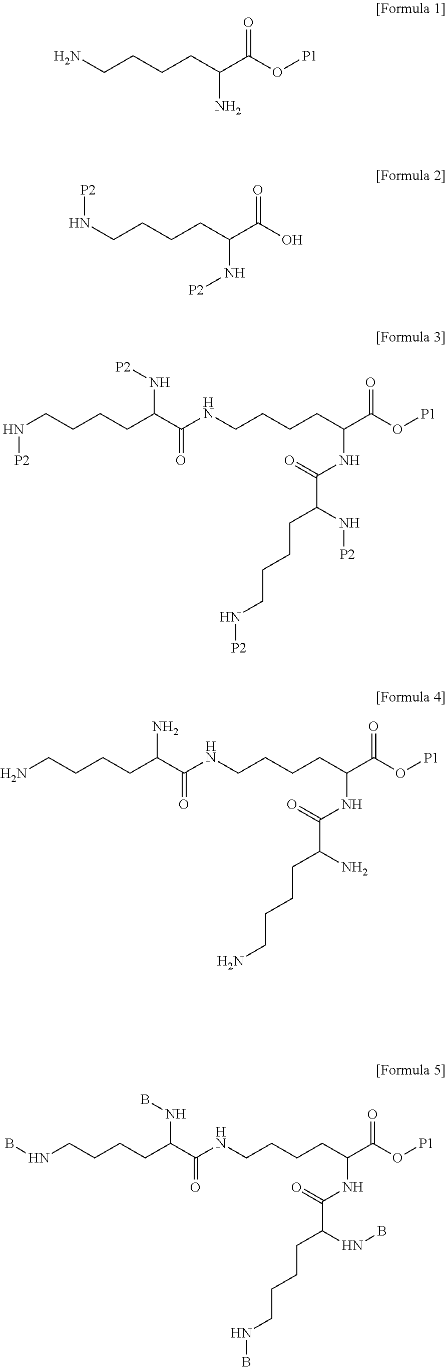 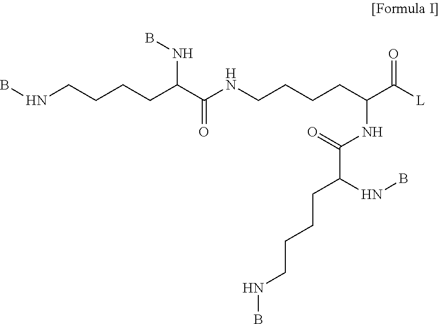
wherein B and L are the same as defined in claim 1; P1 is a carboxyl protecting group; and P2 is an amine protecting group.

13. The preparation method of claim 12, wherein the P1 is C₁-C₆ alkyl or benzyl.

14. The preparation method of claim 12, wherein the P2 is Boc, Cbz, Moz or Fmoc.

15-19. (canceled)

20. A method of treating or preventing diabetes or an anticoagulant activity-associated disease, the method comprising administering a pharmaceutical composition to a subject in need thereof, the pharmaceutical composition comprising:
the drug delivery conjugated moiety or a pharmaceutically acceptable salt thereof according to claim 1 and a polypeptide or polysaccharide biologically active agent.

21. (canceled)
